(19) United States
(12) Patent Application Publication
(10) Pub. No.: US 2005/0053673 A1
(43) Pub. Date: Mar. 10, 2005

(54) Composition and method for facilitating bone healing

(76) Inventors: Shrirang Netke, San Bruno, CA (US); Waheed M. Roomi, Sunnyvale, CA (US); Vadim Ivanov, Castro Valley, CA (US); Aleksandra Niedzwiecki, San Jose, CA (US); Matthias Rath, Almelo (NL)

Correspondence Address: KENYON & KENYON, One Broadway, New York, NY, 10004, US

(21) Appl. No.: 10/657,019

(22) Filed: Sep. 5, 2003

(51) Int. Cl. A61K 38/43; A61K 31/59; A61K 31/525; A61K 31/51; A61K 31/555; A61K 35/78

(52) U.S. Cl. 424/638; 424/639; 424/601; 424/643; 424/682; 424/702; 424/736; 514/52; 514/184; 514/393; 514/423; 514/167; 514/356; 514/350; 514/251; 514/276; 514/564

(57) ABSTRACT

The present invention provides a nutritional composition effective in faciliating bone healing in human, comprising lysine, proline, ascorbic acid, copper, vitamin B₆. The nutritional composition contains 27-34% wt lysine, 14-16% wt proline, and 42-47% wt ascorbic acid. The nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol. The present invention also provides a method of administering the nutritional composition in human effective in facilitating bone healing.

### FIELD OF THE INVENTION

The present invention generally relates to a method of facilitating bone healing.

### BACKGROUND OF THE INVENTION

Bone is a dynamic living tissue and is continuously being replenished by resorption and deposition of bone matrix. The stability of bone depends upon the underlying connective tissue. Oxlund H. et al. have shown that optimally structured collagen is more important for bone strength than bone compactness and its calcium saturation (Bone 1996; 19:479-84). A concentration of cross-links between collagen strands appeared 30% less in bone affected by osteoporosis. Knot L. et al. have shown that collagen structure and spatial organization of its fiber network is critical for deposition of minerals and compactness in the bone, and that the micro-architecture of collagen determines bone strength (Bone 1998; 22:181-7). Savvas M. et al. have shown that a loss of collagen caused by malnutrition is a major factor in loss of bone mass (J. Obstetr. Gynecol. 1989; 96:1392-4). The anorexic women in the study showed a lowered bone density by 18% in the spine and by 25% in the femur. The decrease in bone mass is associated with a 22% decrease in skin collagen.

The healing process after bone fracture is an orderly process that involves multiple phases including: i) hematoma formation; ii) fibro-cartilaginous callus formation; iii) bony callus formation; and iv) bone remodeling. During the healing process, pluripotential cells in the vicinity of the bone fracture differentiate into osteoblasts and chondrocytes. Osteoblasts originate from osteoid tissues and they lay down collagen fibers. Chondrocytes give rise to hypertrophic chondrocytes that deposit a mineralized matrix to form calcified cartilage, which is then remodeled into compact bone.

Despite advances in orthopaedic techniques, healing of bone fractures is a lengthy process, often requires weeks if not months. A patient often suffers a severe restriction of movement for several weeks. Facilitating bone healing and fracture repair (i.e., reducing healing time) would provide a great relief to the patient. This is particularly desirable in adolescents because individuals in this age group have the lowest compliance with doctor recommendations. While doctors recommend that an adolescent use crutches for a certain period of time, the adolescent will often stop using crutches much earlier than recommended.

U.S. Pat. No. 6,258,778 discloses a method of enhancing bone and cartilage repair by administering angiotensin and its analogues. U.S. Pat. No. 5,502,074 discloses a method of facilitating bone healing using benzothiophenes. The safety of use of these drugs is not established. For example, angiotensin is known to exert potent cardiovascular and renal effects, and its use in patients with heart or renal failure may be limited.

U.S. Pat. No. 6,061,597 discloses the application of resonant frequency stimulation to promote fracture healing. U.S. Pat. No. 6,290,714 discloses a low level laser therapy in treating bone fracture. The effectiveness of these approaches is not uncertain and requires expensive medical office visits and/or computer equipment. None of these methods have been clinically proven.

U.S. Pat. No. 5,232,709 discloses a nutritional supplement having a large dose of calcium in treating bone loss. Administering to a bone-fractured individual with a large dose of calcium would cause mineralization of the bone tissue, rather than supplementing bone collagen. The increased bone mineralization causes further hardening of bone. The affected bone becomes more brittle over time, making it prone to compound fractures and shattering under stress.

There is a long-felt need to provide a safe, convenient, affordable and effective approach to facilitate bone healing (i.e., reduce healing time of bone fractures) in humans.

### SUMMARY OF THE INVENTION

The present invention provides a nutritional composition comprising lysine, proline, ascorbic acid, copper, and vitamin B₆. The nutritional composition is suitable for human use and is effective in facilitating bone healing.

Preferably, the nutritional composition contains 27-34% wt lysine, 14-16% wt proline, and 42-47% wt ascorbic acid.

Preferably, the nutritional composition is administered orally. Preferably, the recommended amount is 1,010 mg-8 gram lysine, 560 mg-4 gram proline, 1,500 mg-9 gram ascorbic acid, 2 μg-6 mg copper, and 0.5 mg-10 mg vitamin B₆. More preferably, a recommended amount is 230 mg-10 gram lysine, 120 mg-5 gram proline, 360 mg-15 gram ascorbic acid, 1.5 μg-20 mg copper, and 0.2 mg-20 mg vitamin B₆. Most preferably, a recommended amount is 1,010 mg lysine, 560 mg proline, 1,500 mg ascorbic acid, 330 μg copper and 10 mg vitamin B₆.

Preferably, the nutritional composition is a daily dosage (based on a human subject of average body weight of 72 kg) of 3.2-139 mg/kg lysine, 1.7-69.4 mg/kg proline, 5-208.3 mg/kg ascorbic acid, 0.02-278 μg/kg copper, 2.78-279 μg/kg vitamin B₆.

More preferably, the nutritional composition is a daily dosage of 14-111 mg/kg lysine, 7.8-55.6 mg/kg proline, 20.8-125 mg/kg ascorbic acid, 0.03-83.3 μg/kg copper, and 6.94-139 μg/kg vitamin B₆.

Most preferably, nutritional composition is a daily dosage of 14 mg/kg lysine, 7.8 mg/kg proline, 20.8 mg/kg ascorbic acid, 4.6 μg/kg copper, 139 μg/kg vitamin B₆.

Preferably, nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol.

Preferably, the recommended amount is 67 μg-100 mg vitamin A, 0.7 μg-50 μg vitamin D₃, 0.7 μg-50 μg vitamin E, 1.4 mg-8 mg vitamin B₁, 1.4 mg-8 mg vitamin B₂, 9 mg-250 mg niacin, 18 μg-500 μg folic acid, 4 μg-100 μg vitamin B₁₂, 13 μg-400 μg biotin, 8 mg-100 mg pantothenic acid, 7 mg-40 mg calcium, 3 mg-300 mg phosphorus, 40 mg-200 mg magnesium, 0.5 mg-10 mg zinc, 20 μg-300 μg selenium, 0.8 mg-15 mg manganese, 2 μg-200 μg chromium, 0.8 μg-100 μg molybdenum, 4 mg-300 mg potassium, 20 mg-500 mg citrus fruit peel bioflavanoids, 10 mg-500 mg arginine, 10 mg-400 mg cysteine, 5 mg-400 mg inositol, 5 mg-400 mg carnitine, 1.6 mg-70 mg coenzyme Q₁₀, and 1.6 mg-70 mg pycnogenol.

More preferably, the recommended amount is 166 μg-50 mg vitamin A, 1.65 μg-20 μg vitamin D₃, 1.65 μg-20 μg vitamin E, 3.5 mg-7 mg vitamin B₁, 3.5 mg-7 mg vitamin B₂, 22.5 mg-100 mg niacin, 45 μg-300 μg folic acid, 10 μg-50 μg vitamin B₁₂, 32 μg-300 μg biotin, 20 mg-60 mg pantothenic acid, 17 mg-35 mg calcium, 7 mg-100 mg phosphorus, 50 mg-100 mg magnesium, 3 mg-8 mg zinc, 30 μg-250 μg selenium, 1 mg-3.25 mg manganese, 2 μg-75 μg chromium, 2 μg-75 μg molybdenum, 8 mg-200 mg potassium, 50 mg-250 mg citrus fruit peel bioflavanoids, 100 mg-300 mg arginine, 80 mg-200 mg cysteine, 80 mg-200 mg inositol, 80 mg-200 mg carnitine, 3 mg-35 mg coenzyme Q₁₀, and 3 mg-35 mg pycnogenol.

Most preferably, the recommended amount is 333 μg vitamin A, 3.3 μg vitamin D₃, 3.3 μg vitamin E, 7 mg vitamin B₁, 7 mg vitamin B₂, 45 mg niacin, 90 μg folic acid, 20 μg vitamin B₁₂, 65 μg biotin, 40 mg pantothenic acid, 35 mg calcium, 15 mg phosphorus, 40 mg magnesium, 7 mg zinc, 20 μg selenium, 1.3 mg manganese, 10 μg chromium, 4 μg molybdenum, 20 mg potassium, 100 mg citrus fruit peel bioflavanoids, 40 mg arginine, 35 mg cysteine, 35 mg inositol, 35 mg carnitine, 7 mg coenzyme Q₁₀, and 7 mg pycnogenol.

Preferably, the nutritional composition comprises a daily dosage (based on a human subject of average body weight of 72 kg) of 0.9-1,390 μg/kg vitamin A, 0.01-0.694 μg/kg vitamin D₃, 0.01-0.694 μg/kg vitamin E, 19.4-111 μg/kg vitamin B₁, 19.4-111 μg/kg vitamin B₂, 125-3,472 μg/kg niacin, 0.25-6.94 μg/kg folic acid, 0.05-1.39 μg/kg vitamin B₁₂, 0.181-5.56 μg/kg biotin, 111-1,390 μg/kg pantothenic acid, 97.2-555 μg/kg calcium, 42-4,167 μg/kg phosphorus, 555-2,778 μg/kg magnesium, 6.9-139 μg/kg zinc, 0.28-4.17 μg/kg selenium, 11.1-208.3 μg/kg manganese, 0.03-2.78 μg/kg chromium, 0.01-1.39 μg/kg molybdenum, 55.6-4,167 μg/kg potassium, 278-6.944 μg/kg citrus fruit peel bioflavanoids, 139-6,944 μg/kg arginine, 135-5,555 μg/kg cysteine, 69-5,555 μg/kg inositol, 69-5,555 μg/kg carnitine, 22.2-972 μg/kg coenzyme Q₁₀, and 22.2-972 μg/kg pycnogenol.

More preferably, the nutritional composition comprises a daily dosage (based on a human subject of average body weight of 72 kg) of 2.31-694 μg/kg vitamin A, 0.023-0.278 μg/kg vitamin D₃, 0.023-0.278 μg/kg vitamin E, 48.6-97.2 μg/kg vitamin B₁, 48.6-97.2 μg/kg vitamin B₂, 312.5-3,190 μg/kg niacin, 0.6-4.17 μg/kg folic acid, 0.14-0.69 μg/kg vitamin B₁₂, 0.444-4.17 μg/kg biotin, 278-833 μg/kg pantothenic acid, 236-903 μg/kg calcium, 97.2-1,390 μg/kg phosphorus, 694-1,390 μg/kg magnesium, 41.7-111 μg/kg zinc, 0.42-3.47 μg/kg selenium, 13.9-45.1 μg/kg manganese, 0.07-2.78 μg/kg chromium, 0.03-1.04 μg/kg molybdenum, 111.1-2,778 μg/kg potassium, 694-3,472 μg/kg citrus fruit peel bioflavanoids, 1,389-4,167 μg/kg arginine, 1,111-2,778 μg/kg cysteine, 1,111-2,778 μg/kg inositol, 1,111-2,778 μg/kg carnitine, 41.7-486 μg/kg coenzyme Q₁₀, and 41.7-486 μg/kg pycnogenol.

Most preferably, the nutritional composition comprises a daily dosage (based on a human subject of average body weight of 72 kg) of 4.6 μg/kg vitamin A, 0.046 μg/kg vitamin D₃, 0.046 μg/kg vitamin E, 97.2 μg/kg vitamin B₁, 97.2 μg/kg vitamin B₂, 625 μg/kg niacin, 1.25 μg/kg folic acid, 0.27 μg/kg vitamin B₁₂, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μg/kg selenium, 18.1 μg/kg manganese, 0.14 μg/kg chromium, 0.06 μg/kg molybdenum, 277.8 μg/kg potassium, 1,389 μg/kg citrus fruit peel bioflavanoids, 555 μg/kg arginine, 486 μg/kg cysteine, 486 μg/kg inositol, 486 μg/kg carnitine, 97.2 μg/kg coenzyme Q₁₀, and 97.2 μg/kg pycnogenol. 0.01-0.694 μg/kg vitamin D₃, 18.1 μg/kg manganese, 555 μg/kg arginine, 486 μg/kg cysteine, 4.6 μg/kg vitamin A, 0.046 μg/kg vitamin E, 97.2 μg/kg vitamin B₁, 97.2 μg/kg vitamin B₂, 0.27 μg/kg vitamin B₁₂, 625 μg/kg niacin, 1.25 μg/kg folic acid, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μg/kg selenium, 0.14 μg/kg chromium, 0.06 μg/kg molybdenum, 277.8 μg/kg potassium, 1,389 μg/kg citrus fruit peel bioflavanoids, 486 μg/kg inositol, 486 μg/kg carnitine, 97.2 μg/kg coenzyme Q₁₀, and 97.2 μg/kg pycnogenol.

The present invention provides a method for facilitating bone healing in a mammal, comprising the step of administering to a mammal in need thereof an effective amount of a nutritional composition comprising lysine, proline, ascorbic acid, copper, and vitamin B₆. Preferably, the mammal is a human.

The present invention further provides a method for facilitating bone healing in a mammal comprising the step of administering to a mammal in need thereof an effective amount of a nutritional composition further comprising vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol.

Preferably, the nutritional composition is effective in reducing healing time for bone fractures. Preferably, the healing time is reduced >about 5%. More preferably, the healing time is reduced >about 15%. Most preferably, the healing time is reduced >about 50%.

Preferably, the nutritional composition is effective in humans of all ages. Preferably, the nutritional composition is suitable for facilitating bone healing in adults of 41-40 and 41-50 years of age. The nutritional composition provides a 37% and 40% reduction in healing time respectively. More preferably, the nutritional composition is effective in human of 10-20 years of age (i.e., adolescents), providing a 49% reduction in healing time.

Preferably, the nutritional composition may be administered orally, intravenously, or parenterally.

### BRIEF DESCRIPTION OF THE DRAWINGS

FIG. 1 depicts a radiograph of tibial shaft fracture immediately prior to reduction.

FIG. 2 depicts a radiograph of tibial shaft fracture at healing at 12 weeks.

FIG. 3 depicts ascorbic acid levels (urinalysis) in supplemented (patient #1-29) and placebo (patient #30-70) groups.

FIG. 4 depicts a distribution of patients (percentage) by tibial fracture healing time.

### DETAILED DESCRIPTION OF THE INVENTION

As used herein, the term “bone healing” refers to the healing of bone fractures. Bone healing shall also encompass the process of bone repair and shall not limited to healing of accidental bone fractures. Bone healing also concerns surgical intervention in procedures such as bone replacement (e.g., hip and knee joint replacement) and bone implantation (e.g., tooth implantation). When a bone is healed, normal mobility at the fractured bone site is restored and pain elicited by stressing the fracture or by walking is avoided. General restoration of efficient and painless functioning of the affected limb at the fracture site is provided.

The term “healing time” refers to the time elapsed from the time when bone fracture occurs until the time when the bone fracture is healed. With respect to the experiments performed in the studies disclosed herein, the healing time is measured for the time elapsed from the time of reduction of fractured bone until the bone is healed. “Reduction” refers the process of aligning the tips of a fractured bone (e.g., tibia) at the point of fracture in a position to allow fusing of the fractured bone tips together. “Adolescent” is a human between about 10 and about 20 years of age. “Effective amount” refers to an amount of the present nutritional composition effective in reducing the healing time of bone fracture. “Pharmaceutically acceptable” refers to carriers, diluents, and excipients that are compatible with the other ingredients of the formulation, and not deleterious to the recipient thereof. “% wt” refers to % of a specific ingredient as a % proportion to the total weight of the nutritional composition: for example, “27% wt of lysine” refers to 27% of the total weight of the nutritional composition is lysine.

The present nutritional composition is suitable for use in a mammal. Preferably, the mammal is a human.

Different age groups of humans may exhibit different speeds of bone healing. For example, humans of elder age may more easily suffer from bone fracture (due to decalcification by osteoporosis) and is likely to have a longer healing time. The present nutritional composition is found to be effective in faciliating bone healing in humans in general; and its effectiveness is not limited to a particular age group.

The present invention provides a nutritional composition for facilitating bone healing in humans, preferably in adolescent individuals, comprising the step of administering to a human in need of treatment an effective amount of the composition comprising lysine, proline, ascorbic acid, copper, and vitamin B₆. Preferably, the nutritional composition contains 27-34 % wt lysine, 42-47% wt ascorbic acid and 14-16% wt proline.

The present nutritional composition also contains lysine and proline. Lysine and proline are constituents of collagen and proteins in the bone. Lysine and proline may contribute to osteoblast proliferation of alkaline phosphatase, nitric oxide, insulin-like growth factor-I, and collagen type I and may be essential for proper bone formation.

Lysine may include lysine salts such as hydroxylysine and hydroxylysine salts. A daily dose of 3.2-139 mg/kg lysine is recommended. Preferably, 14 to 111 mg/kg lysine is used; and more preferably, 14 mg/kg lysine is used. For an average individual weighing 72 kg, the daily recommended dosage of lysine is 230 mg to 10 grams; preferably, 1,010 mg to 8 grams; and more preferably 1,010 mg.

Proline is a non-essential amino acid. However, its synthesis in human body could be limited under certain conditions. It has been reported that the stress of fracture lowers non-essential amino acid levels in plasma of older humans. In such a case, deficiency of proline, if any, would adversely affect the healing of fracture, since this amino acid is present in large proportion in collagen.

Proline may include proline salts such as hydroxyproline and hydroxyproline salts. A daily dose of 1.7-69.4 mg/kg proline is recommended. Preferably, 7.8 to 56 mg/kg is used; and more preferably, 7.8 mg/kg is used. For an average individual weighing 72 kg, the daily recommended dosage of proline is 120 mg to 5 grams; preferably, 560 mg to 4 grams; and more preferably 560 mg.

The present nutritional composition contains ascorbic acid. Ascorbic acid may promote the progressive development of osteoblast phenotype and facilitate bone healing and it is also necessary for the differentiation and proliferation of osteogenic and chondrogenic cells.

Ascorbic acid and vitamin C are used interchangeably and may include calcium ascorbate, magnesium ascorbate or ascorbyl palmitate. A daily dose of 5-208 mg/kg ascorbic acid is recommended. Preferably, 20.8-125 mg/kg is used; and more preferably, 20.8 mg/kg is used. For an average individual weighing 72 kg, the daily recommended dosage of ascorbic acid is 360 mg to 15 grams; preferably, 1,500 mg to 9 grams; and more preferably 1,500 mg.

The present invention further provides minerals and/or trace elements. Trace elements may help to catalyze the production of those macromolecules needed for connective tissue structure and function.

Copper, as a cofactor for lysyl oxidase, is essential for intra- and intermolecular cross-links in collagen. Copper deficit has been shown to impair the mechanical strength of bone. It was hypothesized that a relatively large quantity of ascorbic acid, vitamin B₆, L-lysine and L-proline, together with copper, would have a pronounced effect on bone collagen health and function to produce a marked difference in healing time between fractured bones of a supplement group and a placebo group.

Copper compounds may include copper glycinate. A daily dose of 0.02 to 278 μg/kg copper is recommended. Preferably, 0.03 to 83 μg/kg is used; and more preferably, 4.6 μg/kg is used. For an average individual weighing 72 kg, the daily recommended dosage of 1.5 μg to 20 mg; preferably 2 μg to 6 mg; and more preferably, 330 μg.

Vitamin B₆ is of importance in bone healing, as it is instrumental in providing reducing equivalent necessary for mineralization. Vitamin B₆ deficiency caused marked diminution in glucose 6-phosphate dehydrogenase activity in perisoteal bone formation and in developing callus. It also caused changes in the bone suggestive of imbalance between osteoblasts and osteoclasts.

Vitamin B₆ compounds may include pyridoxine HCl. A daily dose of 2.8 to 279 μg/kg vitamin B₆ is recommended. Preferably, 7 to 139 μg/kg vitamin B₆ is used; and more preferably, 139 μg/kg is used. For an average individual weighing 72 kg, the daily recommended dosage of vitamin B₆ is 0.2 to 20 mg; preferably, 0.5 to 10 mg; and more preferably, 10 mg.

Certain ingredients of the present nutritional composition are present at a high amount. Specifically, lysine is present between 27-34% wt (preferably at 28-33% wt); proline is present between 14-16% wt (preferably 15-16% wt); and ascorbic acid is present between 42-47% wt (preferably at 43-46% wt).

Not wishing to be bound by theory, it is believed that the high amounts of these ingredients (i.e., 27-34 wt % lysine, 14-16 wt % proline and 42-47 wt % ascorbic acid), either independently or synergistically act to facilitate bone healing (i.e., reducing healing time) of bone fractures in humans, particularly in adolescents. The present nutritional composition may exert its effect at the structure and function of the bone collagen to promote bone healing.

A recommended daily oral dosage includes 3.2-139 mg/kg lysine, 1.37-69 mg/kg proline, 5-208 mg/kg ascorbic acid, 2.78-279 μg/kg vitamin B₆, and 0.02-278 μg/kg copper. Preferably, the recommended daily oral dosage is: 14-111 mg/kg lysine, 7.8-56 mg/kg proline, 20.8-125 mg/kg ascorbic acid, 6.9-139 μg/kg vitamin B₆, and 0.03-83 μg/kg copper. More preferably, the recommended daily oral dosage is: 14 mg/kg lysine, 7.8 mg/kg proline, 20.8 mg/kg ascorbic acid, 139 μg/kg vitamin B₆, 4.6 μg/kg copper. Preferably, the nutritional composition is administered 3 tablets per day (i.e., one tablet in morning, one tablet in afternoon and one tablet at night).

Several other dietary components, including protein, calcium, magnesium, zinc, copper, iron, fluoride, and vitamins D, A and K, are required for normal bone metabolism. All of these nutrients impact fracture healing, some more directly than others. The trauma of bone fracture was shown to cause a decrease in copper, manganese, and zinc levels in liver, suggesting increased requirement of these minerals after bone fracture.

The present nutritional composition may further comprise vitamin D₃, manganese, arginine, cysteine, vitamins A, E, B₁, B₂, B₁₂, niacin, folic acid, biotin, pantothenic acid, calcium, phosphorus, magnesium, zinc, selenium, chromium, molybdenum, potassium, citrus fruit peel bioflavanoids, inositol, carnitine, coenzyme Q₁₀, and pycnogenol.

The particular dosage of the present nutritional composition requires to facilitate bone healing (i.e., reducing healing time) will depend on the severity of the medical condition, the route of administration and the particular subject being treated. The nutrition composition of the present invention may be administered by a variety of routes including oral, intravenous, or parenteral administration. Preferably, the nutritional composition is in unit dosage form, e.g. tablets or capsules. Thus, the present nutritional composition is recommended to be administered orally as a tablet.

A daily recommended dosage (based on a human subject of average body weight of 72 kg) may further contain 0.9-1,390 μg/kg vitamin A, 0.01-0.694 μg/kg vitamin D₃, 0.01-0.694 μg/kg vitamin E, 19.4-111 μg/kg vitamin B₁, 19.4-111 μg/kg vitamin B₂, 125-3,472 μg/kg niacin, 0.25-6.94 μg/kg folic acid, 0.05-1.39 μg/kg vitamin B₁₂, 0.181-5.56 μg/kg biotin, 111-1,390 μg/kg pantothenic acid, 97.2-555 μg/kg calcium, 42-4,167 μg/kg phosphorus, 555-2,778 μg/kg magnesium, 6.9-139 μg/kg zinc, 0.28-4.17 μg/kg selenium, 11.1-208.3 μg/kg manganese, 0.03-2.78 μg/kg chromium, 0.01-1.39 μg/kg molybdenum, 55.6-4,167 μg/kg potassium, 278-6.944 μg/kg citrus fruit peel bioflavanoids, 139-6,944 μg/kg arginine, 135-5,555 μg/kg cysteine, 69-5,555 μg/kg inositol, 69-5,555 μg/kg carnitine, 22.2-972 μg/kg coenzyme Q₁₀, and 22.2-972 μg/kg pycnogenol.

Preferably, the daily recommended dosage (based on a human subject of average body weight of 72 kg) may further contain 2.31-694 μg/kg vitamin A, 0.023-0.278 μg/kg vitamin D₃, 0.023-0.278 μg/kg vitamin E, 48.6-97.2 μg/kg vitamin B₁, 48.6-97.2 μg/kg vitamin B₂, 312.5-3,190 μg/kg niacin, 0.6-4.17 μg/kg folic acid, 0.14-0.69 μg/kg vitamin B₁₂, 0.444-4.17 μg/kg biotin, 278-833 μg/kg pantothenic acid, 236-903 μg/kg calcium, 97.2-1,390 μg/kg phosphorus, 694-1,390 μg/kg magnesium, 41.7-111 μg/kg zinc, 0.42-3.47 μg/kg selenium, 13.9-45.1 μg/kg manganese, 0.07-2.78 μg/kg chromium, 0.03-1.04 μg/kg molybdenum, 111.1-2,778 μg/kg potassium, 694-3,472 μg/kg citrus fruit peel bioflavanoids, 1,389-4,167 μg/kg arginine, 1,111-2,778 μg/kg cysteine, 1,111-2,778 μg/kg inositol, 1,111-2,778 μg/kg carnitine, 41.7-486 μg/kg coenzyme Q₁₀, and 41.7-486 μg/kg pycnogenol.

Most preferably, the nutritional composition may further include a daily dosage (based on a human subject of average body weight of 72 kg) of 4.6 μg/kg vitamin A, 0.046 μg/kg vitamin D₃, 0.046 μg/kg vitamin E, 97.2 μg/kg vitamin B₁, 97.2 μg/kg vitamin B₂, 625 μg/kg niacin, 1.25 μg/kg folic acid, 0.27 μg/kg vitamin B₁₂, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μg/kg selenium, 18.1 μg/kg manganese, 0.14 μg/kg chromium, 0.06 μg/kg molybdenum, 277.8 μg/kg potassium, 1,389 μg/kg citrus fruit peel bioflavanoids, 555 μg/kg arginine, 486 μg/kg cysteine, 486 μg/kg inositol, 486 μg/kg carnitine, 97.2 μg/kg coenzyme Q₁₀, and 97.2 μg/kg pycnogenol. 0.01-0.694 μg/kg vitamin D₃, 18.1 μg/kg manganese, 555 μg/kg arginine, 486 μg/kg cysteine, 4.6 μg/kg vitamin A, 0.046 μg/kg vitamin E, 97.2 μg/kg vitamin B₁, 97.2 μg/kg vitamin B₂, 0.27 μg/kg vitamin B₁₂, 625 μg/kg niacin, 1.25 μg/kg folic acid, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μg/kg selenium, 0.14 μg/kg chromium, 0.06 μg/kg molybdenum, 277.8 μg/kg potassium, 1,389 μg/kg citrus fruit peel bioflavanoids, 486 μg/kg inositol, 486 μg/kg carnitine, 97.2 μg/kg coenzyme Q₁₀, and 97.2 μg/kg pycnogenol.

The present nutritional composition may include a pharmaceutically acceptable carrier, diluent, or excipient. The nutritional composition of the present invention can be prepared by procedures known in the art. Respective ingredients may be formulated with common excipients, diluents, or carriers, and formed into tablets, capsules, suspensions, powders, and the like. Examples of excipients, diluents, and carriers include: i) fillers and extenders such as starch, sugars, mannitol, and silicic derivatives; ii) binding agents such as carboxymethyl cellulose and other cellulose derivatives, alginates, gelatin, and polyvinyl-pyrrolidone; iii) moisturizing agents such as glycerol; disintegrating agents such as calcium carbonate and sodium bicarbonate; agents for retarding dissolution such as paraffin; iv) resorption accelerators such as quaternary ammonium compounds; v) surface active agents such as acetyl alcohol, and glycerol monostearate; v) adsorptive carriers such as kaolin and bentonite; and vi) lubricants such as talc, calcium and magnesium stearate, and solid polyethyl glycols.

The nutritional compositions may also be formulated as elixirs or solutions for convenient oral administration or as solutions appropriate for parenteral administration, for example, by intramuscular, subcutaneous or intravenous routes. Ideally the formulation is in the form of a pill, tablet, capsule, lozenge, liquid or similar dosage form. The nutritional compositions may well be suited to formulation as sustained release dosage forms and the like.

### Experiments

Tablet Preparation

The ingredients listed in Table 1 were formulated to form tablets. The tablets contained the key ingredients of lysine (1,010 mg), proline (560 mg), ascorbic acid (1,500 mg), copper (330 μg) and vitamin B₆ (10 mg).

The tablets further contained additional ingredients including vitamin D₃, manganese, arginine, cysteine, vitamins A, E, B₁, B₂, B₁₂, niacin, folic acid, biotin, pantothenic acid, calcium, phosphorus, magnesium, zinc, selenium, chromium, molybdenum, potassium, citrus fruit peel bioflavanoids, inositol, carnitine, coenzyme Q₁₀, and pycnogenol.

TABLE 1
Serving Size - Three Tablets/Day

| | | | Daily Dosage per Body | |
|---|---|---|---|---|
| | Daily Dosage | | Weight* | |
| Key Ingredients | | | | |
| L-Lysine (from L-Lysine HCl) | 1,010 | mg (28% wt) | 14.0 | mg/kg |
| L-Proline | 560 | mg (16% wt) | 7.8 | mg/kg |
| Ascorbic Acid (Ascorbyl Palmitate, | 1,500 | mg (42.8% wt) | 20.8 | mg/kg |
| Calcium Ascorbate, Magnesim | | | | |
| Ascorbate) | | | | |
| Copper (Copper Glycinate) | 330 | μg (<0.01% wt) | 4.58 | μg/kg |
| Vitamin B₆ (Pyridoxine HCl) | 10 | mg (0.28% wt) | 139 | μg/kg |
| Additional Ingredients | | | | |
| Vitamin A (7.5% Betatene (Henkel)) | 333 | μg | 4.6 | μg/kg |
| Vitamin D₃ (Cholecalciferol) | 3.3 | μg | 0.046 | μg/kg |
| Vitamin E (Mixed Covitol) | 3.3 | μg | 0.046 | μg/kg |
| Vitamin B₁ (Thiamine Mononitrate) | 7 | mg | 97.2 | μg/kg |
| Vitamin B₂ (Riboflavin) | 7 | mg | 97.2 | μg/kg |
| Niacin (Niacinamide) | 45 | mg | 625 | μg/kg |
| Folic Acid | 90 | μg | 1.25 | μg/kg |
| Vitamin B₁₂ (Cyanocobalamin) | 20 | μg | 0.27 | μg/kg |
| Biotin | 65 | μg | 0.90 | μg/kg |
| Pantothenic Acid (D-Calcium | 40 | mg | 555 | μg/kg |
| Pantothenate) | | | | |
| Calcium (Gycinate, Ascorbate) | 35 | mg | 486 | μg/kg |
| Phosphorus (Dicalcium Phosphate) | 15 | mg | 208 | μg/kg |
| Magnesium (Magnesium Glycinate, | 40 | mg | 555 | μg/kg |
| Magnesium Ascorbate) | | | | |
| Zinc (Zinc Glycinate) | 7 | mg | 97.2 | μg/kg |
| Selenium (L-Selenomethionine) | 20 | μg | 0.78 | μg/kg |
| Manganese (Amino Acid Chelate) | 1.3 | mg | 18.1 | μg/kg |
| Chromium (Chromium Glycanate) | 10 | μg | 0.14 | μg/kg |
| Molybdenum (Molybdenum Glycinate) | 4 | μg | 0.06 | μg/kg |
| Potassium (Potassium Proteinate) | 20 | mg | 277.8 | μg/kg |
| Citrus Fruit Peel Bioflavanoids | 100 | mg | 1,389 | μg/kg |
| L-Arginine (L-Arginine HCl) | 40 | mg | 555 | μg/kg |
| L-Cysteine (L-Cysteine Monohydrate | 35 | mg | 486 | μg/kg |
| HCl) | | | | |
| Inositol | 35 | mg | 486 | μg/kg |
| L-Carnitine (L-Carnitine Tartrate) | 35 | mg | 486 | μg/kg |
| CoEnzyme Q₁₀ | 7 | mg | 97.2 | μg/kg |
| Pycnogenol | 7 | mg | 97.2 | μg/kg |
| TOTAL | 3.5 | g | 48.6 | mg/kg |

*Body Weight refers to a human subject of average body weight of 72 kg

### Clinical Studies

Patient Selection:

A randomized double-blind placebo-controlled study was performed. The clinical study was conducted according to the recommendations of the Declaration of Helsinki, its amendments and the AMG. The inclusion criteria for patient admission were: (i) unilateral displaced closed or grade I open fractures of tibial shaft; and (ii) age above 10 years. The exclusion criteria for patient admission were: (i) patients who had other major injuries, (ii) patients with cardiopulmonary, rheumatological, neurological or metabolic diseases, (iii) patients with previous injuries which influenced their general functions, (iv) patients with fractures within 5 cm distal to tibial tuberosity or within 5 cm proximal to the ankle joint.

Admitted patients received either standard care and placebo or standard care with supplementation with a nutritional supplement comprising lysine, proline, ascorbic acid, copper and vitamin B₆. Qualifying patients, on admission to the study, were clinically examined and the radiographs of the affected limbs were taken, fractures reduced under anesthesia and above-knee plaster casts applied.

Efforts were made to ensure that age groups and fracture types were equally distributed in the two groups. A total of 113 patients with unilateral displaced closed or grade I open tibial fractures were studied. Admitted patients were given informed consent. Participants were advised that data obtained from the studies would be submitted for publication. Out of these, 54 patients were assigned to the supplemented group and 59 patients were assigned to the placebo group. Table 2 summarizes the age distribution of the total 131 patients.

TABLE 2
Distribution of Patients by Age Groups

| | Age | | | | | |
|---|---|---|---|---|---|---|
| | 10-20 | 21-30 | 31-40 | 41-50 | >50 | |
| | years | years | years | years | years | Total |
| Supplemented | 2 (9.5%)* | 7 (33.3%) | 6 (28.6%) | 3 (14.3%) | 3 (14.3%) | 21 (100%) |
| Group | | | | | | |
| Placebo Group | 8 (22.2%) | 10 (27.8%) | 10 (27.8)% | 4 (11.1%) | 4 (11.1%) | 36 (100%) |

*Values in parentheses represent percentage of total patients in the specified age group

### Clinical Protocol

All fractures were reduced (closed reduction) under anesthesia and above-knee plaster casts applied. The fractured limbs were routinely radiographed before and after reduction. The supplemented group of patients were supplied with the nutritional composition in Table 1 and the placebo group of patients with bottles containing placebo tablets. The nutritional composition listed in Table 1 was studied to evaluate if the proposed desired supplements can ensure adequacy of nutrients impacting fracture healing. The placebo tablets contained material of no medical significance, such as cellulose, fructose etc., but were physically indistinguishable from nutrient tablets. All patients were asked to take one tablet thrice daily (morning, afternoon and night).

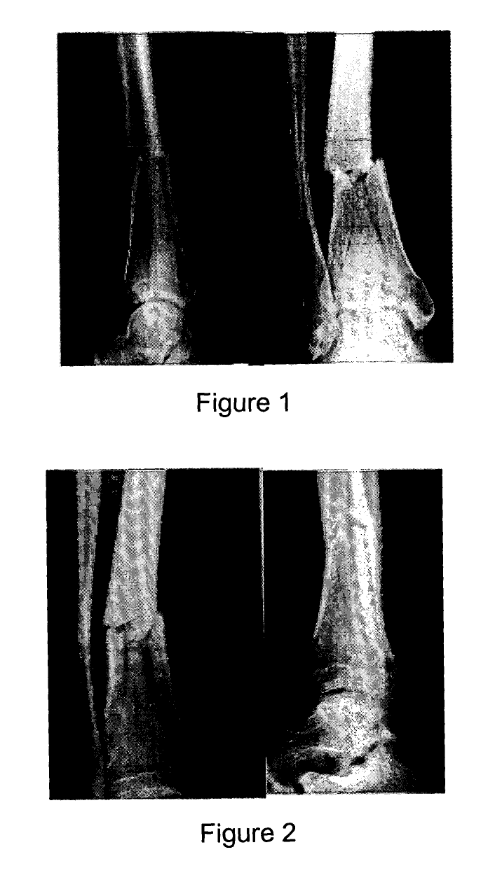

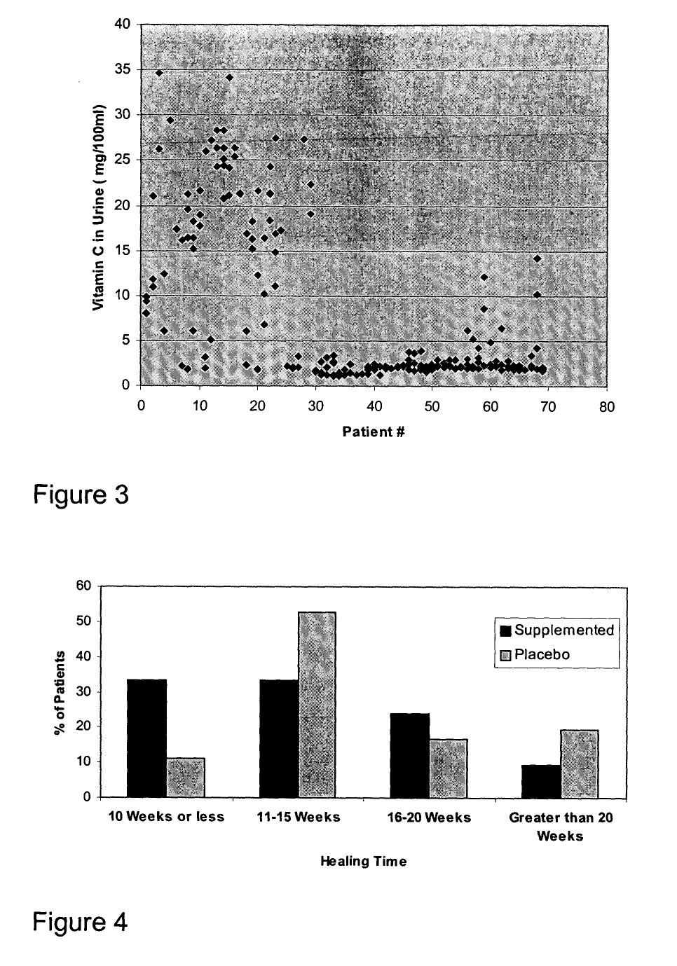

Urine samples of the patients were taken to evaluate their baseline ascorbic acid levels. Blood samples were also taken to assess the baseline calcium levels (FIG. 3). The patients were then discharged from the hospital and were asked to return for check-up every four weeks until the treating orthopedic surgeon deemed the fracture healed. At each follow-up examiantion, tibial fractures were radiographed, and patients were clinically examined and tested for urinary vitamin C and blood calcium levels. The radiological examination was done to confirm that the fragments of the fracture remained in reduced position and that callus formation was progressing satisfactorily. The ascorbic acid content of the urine was determined by spectrophotometry and blood calcium content was determined with commercial kits. Healing was defined as absence of abnormal mobility at the fracture site clinically and absence of pain elicited by stressing the fracture or by walking. Radiographic confirmation of callus formation was used as supporting evidence for healing. (See FIGS. 1 and 2 for radiographic examples). A healing period of greater than 20 weeks without any surgical intervention was considered delayed healing.

### Statistical Analysis

The results were expressed as means±standard error for the groups. The Wilcoxon test was performed on the fracture healing times among various groups. Statistical significance was set at P<0.05.

### Results

29 patients from the supplemented group and 42 patients from the placebo group returned for regular follow-ups until fractures were deemed healed.

Overall, the urinary ascorbic acid content in the supplemented patient group was higher than that in the placebo group (FIG. 3). Low urinary ascorbic acid values (below 5 mg/100 ml of urine) were detected in eight supplemented patients and high ascorbic acid values (above 5 mg/100 ml of urine) in six placebo patients at any one of the check-up visits. These patients were excluded from evaluation. Therefore, only 21 patients in the supplemented group and 36 patients in the placebo group remained for the completion of the study. Blood calcium levels of all participants in the study were within normal limits.

The age distribution of the patients is detailed in Table 2. The age range of the patients in the supplemented group was between 15 and 65 years, with a mean age of 35 years. The age range of the patients in the placebo group was between 12 and 75 years, with a mean age of 32 years.

The healing time of the patients is detailed in Table 4. The mean healing time for the patients in the supplemented group was 14.0±1.1 weeks. The mean healing time for the patients in the placebo group was 16.9±1.2 weeks. These data show that the overall mean healing time in the supplemented group is about three weeks shorter than that for the placebo group (i.e., 17.2%). This difference attains statistical significance at t=1.07, p=0.288.

Percentile classification of the data indicates that in the 75th percentile, fractures in the supplemented group healed within 17 weeks, while those in the placebo group healed in 19 weeks (Table 3).

TABLE 3
Effect of Supplementation on Bone Fracture Healing Time

| | Supplemented | Placebo |
|---|---|---|
| Criteria | Group | Group |
| Number of patients | 21 | 36 |
| Age range (years) | 15 to 65 | 12 to 75 |
| Mean age (years) | 35 | 32 |
| Healing time (weeks) | 14.0 ± 1.1 | 16.9 ± 1.2 |
| 75^{th} percentile healing period (weeks) | 17 | 19 |

The percentage of patients experiencing early fracture healing (in 10 weeks or less) was 33.3% in the supplemented group and 11.1% in the placebo group (Chi-square analysis=2.853, p=0.091). The percentage of patients that had delayed healing (more than 20 weeks) was 9.5% in the supplemented group and 19.4% in the placebo group (Table 4).

TABLE 4
Distribution (Percentage) of Patients by Fracture Healing Time

| Healing Time | Supplemented Group | Placebo Group |
|---|---|---|
| 10 weeks or less | 33.3% | 11.1% |
| 11 to 15 weeks | 33.3% | 52.8% |
| 16 to 20 weeks | 23.8% | 16.7% |
| More than 20 weeks | 9.5% | 19.4% |

The healing time of bone fracture in patients of different ages is shown in Table 6. In the patients 10-20 years of age, the healing time reduced from 17.6 weeks to 9 weeks (reduced 49%). In the patients of 31-40 years of age, the healing time reduced from 17.1 weeks to 10.7 weeks (reduced 37%). In the patients 41-50 years of age, the healing time reduced from 21.2 weeks to 12.7 weeks (reduced 40%). In the patients >50 years of age, the healing time reduced from 16 weeks to 15.7 weeks (reduced 1.8%). Overall, the healing time in the supplemented group, except in the 21-30 year old group, is reduced. (Table 5).

TABLE 5
Healing Time (in Weeks) for Various Age Groups

| | Age Groups | | | | |
|---|---|---|---|---|---|
| | 10-20 yr | 21-30 yr | 31-40 yr | 41-50 yr | >50 yr |
| Supplemented | | | | | |
| Group | | | | | |
| Healing Time | | | | | |
| Mean (in wks) | 9 | 18.3 | 10.7 | 12.7 | 15.7 |
| Range (in wks) | 9 | 9-26 | 6-16 | 11-14 | 14-19 |
| Placebo Group | | | | | |
| Healing Time | | | | | |
| Mean (in wks) | 17.6 | 14.6 | 17.1 | 21.2 | 16 |
| Range (in wks) | 9-30 | 9-30 | 11-39 | 13-30 | 10-20 |

The data suggest that administering to patients suffering from bone fracture with the present nutritional composition at the specified doses effectively reduces the healing time by at least two weeks in 75% of the patients. Patients in the supplemented group also reported an enhanced feeling of general well being during the study. The strongest effects can be seen in the adolescent age group (i.e., 10 to 20 years of age) which had a 49% reduction in the healing time. Patients 41-50 years and 31-40 years of age also had a significant reduction in the healing time (i.e., 40% and 37%, respectively). It is believed that patients in other age groups may likely to receive the same benefits if the dosage of the nutritional supplementation is optimized.

There was no signficant difference in the blood calcium levels in the supplemented group as compared to those in the placebo group. All the patients were found to have their blood calcium levels within the normal range.

The reduction in healing time is believed to be due to the supplementation of key ingredients of which comprises lysine, proline, ascorbic acid, copper and vitamin B₆, as well as additional ingredients used in the present study. The present data provide evidence that nutritional supplementation in patients suffering from bone fracture can facilitate the healing (i.e., reduce healing time). Administration of the present nutritional compositions to bone fractured patients would have a positive impact, early functional recovery, improved well being, reduced medical costs, and reduced cost to business.

It will be understood that there is no intent to limit the present invention to the preferred embodiment disclosed, but rather it is intended to cover all modifications and alternate constructions falling within the spirit and scope of the invention. All publications and other references mentioned herein are incorporated by reference in their entirety.

1. A nutritional composition suitable for facilitating bone healing in a mammal, comprising lysine, proline, ascorbic acid, copper, and vitamin B₆.

2. The nutritional composition of claim 1, wherein the nutritional composition comprises 230 mg-10 grams lysine, 120 mg-5 grams proline, 360 mg-15 grams ascorbic acid, 1.5 μg-20 mg copper, and 0.2 mg-20 mg vitamin B₆.

3. The nutritional composition of claim 1, wherein the nutritional composition comprises 1,010 mg-8 grams lysine, 560 mg-4 grams proline, 1,500 mg-9 grams ascorbic acid, 2 μg-6 mg copper, and 0.5 mg-10 mg vitamin B₆.

4. The nutritional composition of claim 1, wherein the nutrition composition comprises 1,010 mg lysine, 560 mg proline, 1,500 mg ascorbic acid, 330 μg copper and 10 mg vitamin B₆.

5. The nutritional composition of claim 1, wherein the nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol.

6. The nutritional composition of claim 5, wherein the nutritional composition comprises 67 μg-100 mg vitamin A, 0.7 μg-50 μg vitamin D₃, 0.7 μg-50 μg vitamin E, 1.4 mg-8 mg vitamin B₁, 1.4 mg-8 mg vitamin B₂, 9 mg-250 mg niacin, 18 μg-500 μg folic acid, 4 μg-100 μg vitamin B₁₂, 13 μg-400 μg biotin, 8 mg-100 mg pantothenic acid, 7 mg-40 mg calcium, 3 mg-300 mg phosphorus, 40 mg-200 mg magnesium, 0.5 mg-10 mg zinc, 20 μg-300 μg selenium, 0.8 mg-15 mg manganese, 2 μg-200 μg chromium, 0.8 μg-100 μg molybdenum, 4 mg-300 mg potassium, 20 mg-500 mg citrus fruit peel bioflavanoids, 10 mg-500 mg arginine, 10 mg-400 mg cysteine, 5 mg-400 mg inositol, 5 mg-400 mg carnitine, 1.6 mg-70 mg coenzyme Q₁₀, and 1.6 mg-70 mg pycnogenol.

7. The nutritional composition of claim 5, wherein the nutritional composition comprises 166 μg-50 mg vitamin A, 1.65 μg-20 μg vitamin D₃, 1.65 μg-20 μg vitamin E, 3.5 mg-7 mg vitamin B₁, 3.5 mg-7 mg vitamin B₂, 22.5 mg-100 mg niacin, 45 μg-300 μg folic acid, 10 μg-50 μg vitamin B₁₂, 32 μg-300 μg biotin, 20 mg-60 mg pantothenic acid, 17 mg-35 mg calcium, 7 mg-100 mg phosphorus, 50 mg-100 mg magnesium, 3 mg-8 mg zinc, 30 μg-250 μg selenium, 1 mg-3.25 mg manganese, 2 μg-75 μg chromium, 2 μg-75 μg molybdenum, 8 mg-200 mg potassium, 50 mg-250 mg citrus fruit peel bioflavanoids, 100 mg-300 mg arginine, 80 mg-200 mg cysteine, 80 mg-200 mg inositol, 80 mg-200 mg carnitine, 3 mg-35 mg coenzyme Q₁₀, and 3 mg-35 mg pycnogenol.

8. The nutritional composition of claim 5, wherein the nutritional composition comprises 333 μg vitamin A, 3.3 μg vitamin D₃, 3.3 μg vitamin E, 7 mg vitamin B₁, 7 mg vitamin B₂, 45 mg niacin, 90 μg folic acid, 20 μg vitamin B₁₂, 65 μg biotin, 40 mg pantothenic acid, 35 mg calcium, 15 mg phosphorus, 40 mg magnesium, 7 mg zinc, 20 μg selenium, 1.3 mg manganese, 10 μg chromium, 4 μg molybdenum, 20 mg potassium, 100 mg citrus fruit peel bioflavanoids, 40 mg arginine, 35 mg cysteine, 35 mg inositol, 35 mg carnitine, 7 mg coenzyme Q₁₀, and 7 mg pycnogenol.

9. The nutritional composition of claims 1 or 5, wherein the nutritional composition contains 27-34% wt lysine, 14-16% wt proline, and 42-47% wt ascorbic acid.

10. The nutritional composition of claims 1 or 5, wherein the mammal is a human.

11. A method for facilitating bone healing in a mammal, comprising the step of administering to a mammal in need thereof an effective amount of a nutritional composition comprising lysine, proline, ascorbic acid, copper, and vitamin B₆.

12. The method of claim 11, wherein the effective amount of the nutritional composition is a daily dosage of 3.2-139 mg/kg lysine, 1.7-69.4 mg/kg proline, 5-208.3 mg/kg ascorbic acid, 0.02-278 μg/kg copper, 2.78-279 μg/kg vitamin B₆.

13. The method of claim 11, wherein the effective amount of the nutritional composition is a daily dosage of 14-111 mg/kg lysine, 7.8-55.6 mg/kg proline, 20.8-125 mg/kg ascorbic acid, 0.03-83.3 μg/kg copper, and 6.94-139 μg/kg vitamin B₆.

14. The method of claim 11, wherein the effective amount of the nutritional composition is a daily dosage of 14 mg/kg lysine, 7.8 mg/kg proline, 20.8 mg/kg ascorbic acid, 4.6 μg/kg copper, 139 μg/kg vitamin B₆.

15. The method of claim 11, wherein the nutritional composition contains 27-34% wt lysine, 14-16% wt proline, and 42-47% wt ascorbic acid.

16. The method of claim 11, wherein the nutritional composition further comprises vitamin A, vitamin D₃, vitamin E, vitamin B₁, vitamin B₂, niacin, folic acid, vitamin B₁₂, biotin, pantothenic acid, calcium, phosphorus, magnesium, zinc, selenium, manganese, chromium, molybdenum, potassium, citrus fruit peel bioflavanoids, arginine, cysteine, inositol, carnitine, coenzyme Q₁₀, and pycnogenol.

17. The method of claim 11, wherein the nutritional composition further comprises 0.9-1,390 μg/kg vitamin A, 0.01-0.694 μg/kg vitamin D₃, 0.01-0.694 μg/kg vitamin E, 19.4-111 μg/kg vitamin B₁, 19.4-111 μg/kg vitamin B₂, 125-3,472 μg/kg niacin, 0.25-6.94 μg/kg folic acid, 0.05-1.39 μg/kg vitamin B₁₂, 0.181-5.56 μg/kg biotin, 111-1,390 μg/kg pantothenic acid, 97.2-555 μg/kg calcium, 42-4,167 μg/kg phosphorus, 555-2,778 μg/kg magnesium, 6.9-139 μg/kg zinc, 0.28-4.17 μg/kg selenium, 11.1 -208.3 μg/kg manganese, 0.03-2.78 μg/kg chromium, 0.01-1.39 μg/kg molybdenum, 55.6-4,167 μg/kg potassium, 278-6.944 μg/kg citrus fruit peel bioflavanoids, 139-6,944 μg/kg arginine, 135-5,555 μg/kg cysteine, 69-5,555 μg/kg inositol, 69-5,555 μg/kg carnitine, 22.2-972 μg/kg coenzyme Q₁₀, and 22.2-972 μg/kg pycnogenol.

18. The method of claim 11, wherein the nutritional composition further comprises 2.31-694 μg/kg vitamin A, 0.023-0.278 μg/kg vitamin D₃, 0.023-0.278 μg/kg vitamin E, 48.6-97.2 μg/kg vitamin B₁, 48.6-97.2 μg/kg vitamin B₂, 312.5-3,190 μg/kg niacin, 0.6-4.17 μg/kg folic acid, 0.14-0.69 μg/kg vitamin B₁₂, 0.444-4.17 μg/kg biotin, 278-833 μg/kg pantothenic acid, 236-903 μg/kg calcium, 97.2-1,390 μg/kg phosphorus, 694-1,390 μg/kg magnesium, 41.7-111 μg/kg zinc, 0.42-3.47 μg/kg selenium, 13.9-45.1 μg/kg manganese, 0.07-2.78 μg/kg chromium, 0.03-1.04 μg/kg molybdenum, 111.1-2,778 μg/kg potassium, 694-3,472 μg/kg citrus fruit peel bioflavanoids, 1,389-4,167 μg/kg arginine, 1,111-2,778 μg/kg cysteine, 1,111-2,778 μg/kg inositol, 1,111-2,778 μg/kg carnitine, 41.7-486 μg/kg coenzyme Q₁₀, and 41.7-486 μg/kg pycnogenol.

19. The method of claim 11, wherein the nutritional composition further comprises 4.6 μg/kg vitamin A, 0.046 μg/kg vitamin D₃, 0.046 μg/kg vitamin E, 97.2 μg/kg vitamin B₁, 97.2 μg/kg vitamin B₂, 625 μg/kg niacin, 1.25 μg/kg folic acid, 0.27 μg/kg vitamin B₁₂, 0.9 μg/kg biotin, 555 μg/kg pantothenic acid, 486 μg/kg calcium, 208 μg/kg phosphorus, 555 μg/kg magnesium, 97.2 μg/kg zinc, 0.78 μg/kg selenium, 18.1 μg/kg manganese, 0.14 μg/kg chromium, 0.06 μg/kg molybdenum, 277.8 μg/kg potassium, 1,389 μg/kg citrus fruit peel bioflavanoids, 555 μg/kg arginine, 486 μg/kg cysteine, 486 μg/kg inositol, 486 μg/kg carnitine, 97.2 μg/kg coenzyme Q₁₀, and 97.2 μg/kg pycnogenol.

20. The method of claims 11 or 16, wherein the nutritional composition contains 27-34% wt lysine, 14-16% wt proline, and 42-47% wt ascorbic acid.

21. The method of claims 11 or 16, wherein the mammal is a human.

22. The method of claims 11 or 16, wherein the nutritional composition is effective in reducing >about 5% bone healing time.

23. The nutritional composition of claim 20, wherein the nutritional composition is effective in reducing >about 15% bone healing time.

24. The nutritional composition of claim 20, wherein the nutritional composition is effective in reducing >about 50% bone healing time.

25. The method of claims 11 or 16, wherein the step of administering is performed orally, intravenously or parenterally.

26. The method of claim 21, wherein the step of administering is performed orally.
